(19) United States
(12) Patent Application Publication
(10) Pub. No.: US 2023/0416973 A1
(43) Pub. Date: Dec. 28, 2023

(54) DRYING INSTALLATION

(71) Applicant: Helmut JANNACH, Graz (AT)

(72) Inventor: Helmut JANNACH, Graz (AT)

(21) Appl. No.: 18/039,547

(22) PCT Filed: Dec. 1, 2021
(86) PCT No.: PCT/AT2021/060458

§ 371 Date: May 31, 2023

(30) Foreign Application Priority Data
Dec. 3, 2020 (AT) A51056/2020

(51) Int. Cl. D06F 58/38; D06F 58/10; D06F 58/26; D06F 58/46; A47L 23/20; A61L 2/26; A61L 2/06

(52) U.S. Cl. CPC D06F 58/38; D06F 58/10; D06F 58/26; D06F 58/46; A47L 23/205; A61L 2/26; A61L 2/06; D06F 2105/28

(57) ABSTRACT

The invention relates to a drying installation, in particular for drying objects such as items of clothing and equipment items, wherein the objects can be suspended or placed on drying hangers (11, 12), and wherein the drying hangers (11, 12) are arranged in a thermally insulated, heatable drying cabinet (1) which, in addition, is heatable.

The invention relates to a drying installation, in particular for drying objects such as items of clothing and equipment items wherein the objects are suspendable or placeable on drying hangers.

Such drying installations are known in the state of the art for drying items of clothing. The items of clothing are preferably professional garments such as those for the fire brigade, mining companies, the military, medical companies, etc. The drying installations usually have drying hangers formed of tubes, the shape of which is approximately adapted to the item of clothing to be dried. In one embodiment, the drying hangers have a plurality of air outlet openings through which any heated compressed air passed through the tube can flow and escape from the air outlet openings. This makes it possible to dry items of clothing and emergency clothing such as helmets, masks, etc. from the inside or, due to the air turbulence created at the air outlet openings on the drying hangers, also from the outside. This is particularly necessary for special clothing that is impermeable to water and made of heavy materials. These materials dry slowly unless specific drying devices are used.

Known drying installations often have a rack or a box that is open to the front, with an air distribution duct in the upper area of the rack or box, which on the one hand can supply warm air to the drying hangers and on the other hand further serves as a suspension device for the drying hangers.

Such drying devices usually work with drying air temperatures between 30° C. and 50° C., so that on the one hand, the desired drying effect is achieved and on the other hand, the items of clothing are not damaged by excessively high temperatures.

Experience shows that it may be necessary to expose such items of clothing and associated equipment items to higher temperatures in order to combat and ultimately destroy bacteria, microorganisms and viruses. This is intended to ensure the safety of the wearer of the item of clothing and the entire environment, even if, for example, work is carried out in situations of high viral load, as can be the case, for example, with medical staff and rescue personnel, but also with fire brigades.

The present invention solves the problem of achieving the necessary higher temperature in a drying installation, which should usually be between 60° C. and 80° C., and still being able to carry out drying at a lower air temperature. A temperature up to 80° C. would be too high for the dry air temperature to be introduced into the items of clothing.

According to the present invention, the drying hangers are arranged in a thermally insulated drying cabinet, which, in addition, is heatable. Thus, the items of clothing and equipment items can be hung in the drying cabinet attached to the drying hanger, which can additionally be heated. To save energy, the drying cabinet is thermally insulated.

According to another feature, the drying cabinet is provided with a bottom, a top, left and right side walls, a rear wall and an openable and closable front wall.

Optionally, the drying hangers in the drying cabinet are suppliable or are supplied with warm air by a warm air blower, the drying hangers being provided with one or more air outlet openings along their extent.

Optionally, the warm air blower is arranged outside, preferably on the top or also inside the drying cabinet, and an air duct is led from the warm air blower to an air distribution duct to which the drying hangers inside the drying cabinet are connected or can be connected.

Optionally, a heating device is arranged inside for heating the drying cabinet, preferably in the bottom region or also on the top or on the sides. The heating device may be an electric heater. Optionally, the heating device is a hot air blower.

Optionally, the heating device is configured for a cabinet temperature between 60° C. and ° C., preferably 65° C. to 70° C. The warm air blower is configured for a dry air temperature between 30° C. and 60° C., preferably between 40° C. to 50° C., optionally also up to 80° C.

Optionally, the cabinet temperature is monitored and controlled by a thermostat. A time switch may be provided for the switching times and optionally also for the warm air blower. The time switch may be a component of an electronic controller which is set up to control the switching times for the supply of drying air to the drying hangers and for the heating device and to control the drying air temperatures and cabinet temperature.

The invention is further characterised by a method for drying and decontaminating objects such as items of clothing and equipment items, and preferably by using the drying installation described above.

In the following, the invention is described in more detail by way of example with reference to the figures.

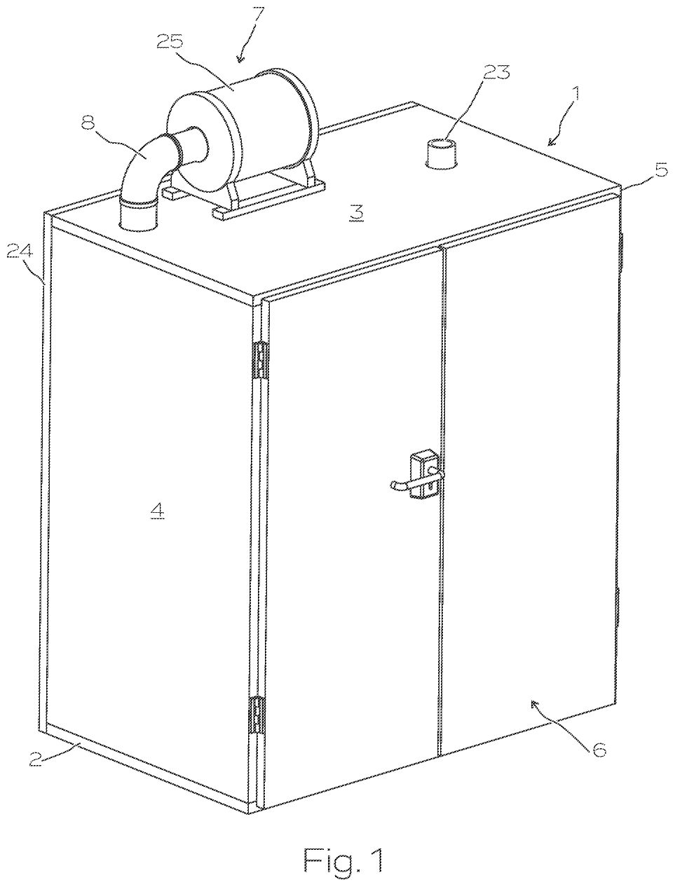

FIG. 1 shows an angled view of the drying cabinet.

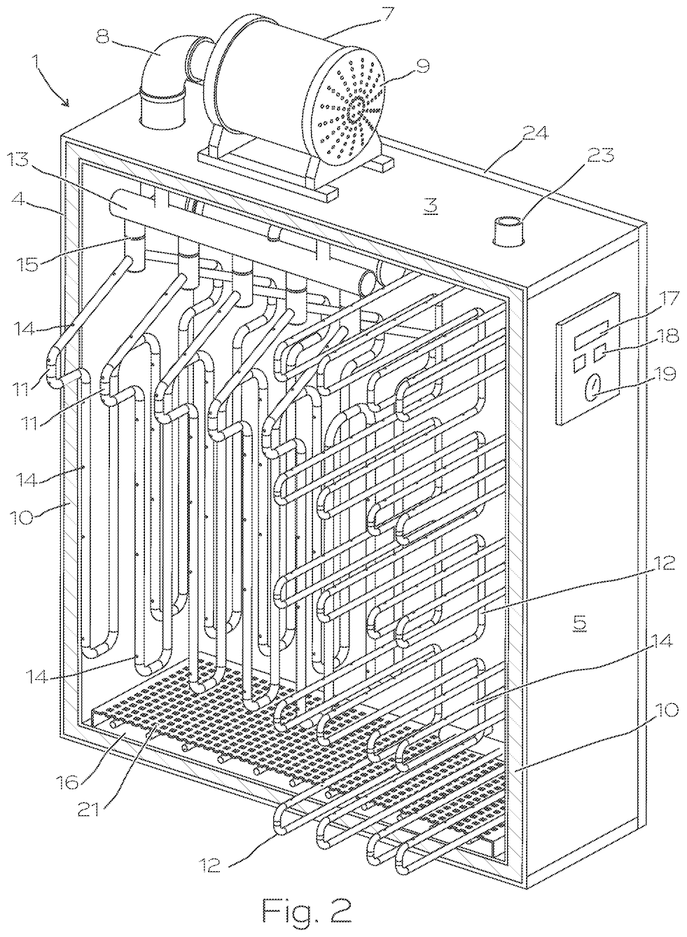

FIG. 2 shows an angled cutaway view of the drying cabinet depicting the inner equipment.

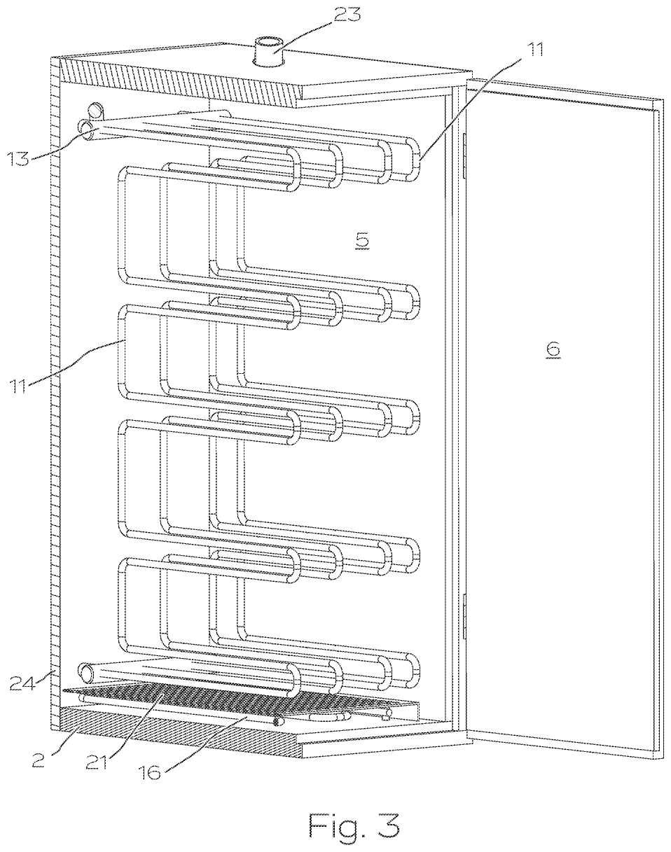

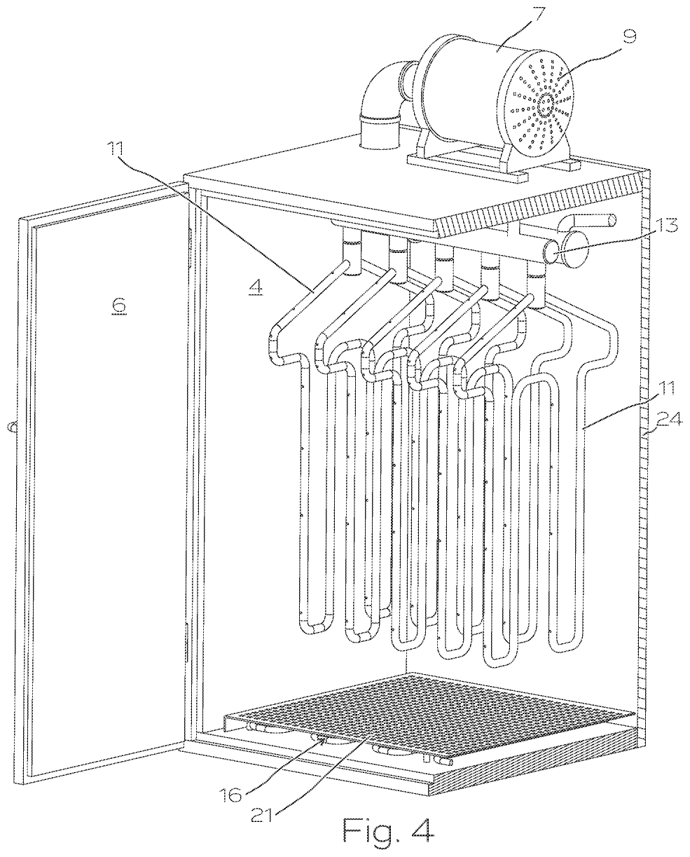

FIGS. 3 and 4 show more details of the two halves of the drying cabinet, showing the bottom region.

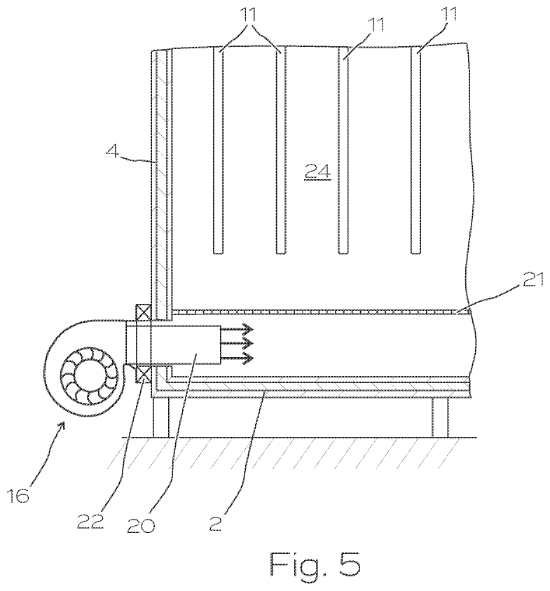

FIG. 5 shows an alternative of the bottom region of a drying cabinet in purely schematic representation.

According to FIG. 1, the drying installation comprises a drying cabinet 1, which substantially comprises a bottom 2, a top 3, left side wall 4 and right side wall 5 and a front wall 6, wherein the front wall 6 comprises two doors, possibly also sliding doors, which are openable and closable. Opening the front wall 6 allows unhindered access to the inside of the drying cabinet 1. The rear wall is indicated with reference sign 24.

FIG. 1 further shows the warm air blower 7, which preferably contains a high-quality electric motor and directs the generated warm air into the interior of the drying cabinet 1 via the air duct 8. Preferably, a warm air blower 7 is used, which not only generates the strong air flow required, but also emits the necessary heat. For this purpose, as shown in FIG. 1, the motor and blower may be enclosed in a housing 25 so that the heat from the motor does not flow away unused but is used to heat the air flow. This design is energy saving and also prevents the ambient air from heating up too much, which is also particularly desirable in staff rooms.

Of course, it is also possible and within the scope of the invention to provide an air blower which is arranged differently, e.g. separately from the drying cabinet 1, and which has a heating source in the air duct 8 to generate the desired temperature of the drying air.

For venting the drying cabinet 1, a vent opening 23 is provided, which is arranged at the top 3, for example, or may also be arranged in a different way.

FIG. 2 shows the drying cabinet 1 from a different angle, showing the warm air blower 7 with the air inlet openings 9. The drying cabinet 1 is shown in a cutaway view to better show the inside of the drying cabinet 1. Furthermore, the insulation 10 for thermal insulation of the drying cabinet 1 is shown.

In the exemplary embodiment according to FIG. 2, two types of drying hangers 11, 12 are exemplarily arranged inside the drying cabinet 1. The drying hangers 11 are shaped tubes suitable for items of clothing and the drying hangers 12 are suitable for holding shoes or equipment items such as harnesses, gloves, helmets and masks etc. This arrangement of two different drying irons is optional. It is also possible to hang just one variant or more different variants of drying hangers.

The drying hangers 11, 12 are fed from above by an air distribution duct 13 with the dry air from the warm air blower 7. The drying hangers 11, 12 have air outlet openings 14 distributed along their extent, through which the drying air can be fed into the inside of the suspended items of clothing or to all sides of the equipment items.

The drying hangers 11 are attached to the air distribution duct 13 by means of a coupling which allows the drying hanger 11 to be easily hooked in and unhooked and allows the drying air to enter.

A heating device 16 is provided in the bottom region of the drying cabinet 1, which in the present exemplary embodiment is formed by electric heating coils. The heating device 16 is covered at the top by a grille 21 so that the warm air can easily flow upwards and heat up the entire content of the drying cabinet. The heating device 16 may also be arranged at other locations inside the drying cabinet 1, for example on the side walls 4, 5 or on the top 3.

Of course, the heating device 16 must be provided with appropriate electrical connections and switching devices, which are only partially shown herein.

In this embodiment, the right side wall 5 accommodates the corresponding control elements for operating the drying installation. Among the elements are, e.g., a temperature display 17, a thermostat 18 with its switching elements and a time switch 19. However, the control elements may also be arranged in the door region or above the door with visible heat monitoring.

The time switch and the associated display and switching elements may be components of an electronic controller, which either is attached to the drying cabinet or may be designed as a portable electronic switching device that is connectable via a plug connection or an interface.

FIGS. 3 and 4 show the left and right halves of the drying cabinet 1 respectively, the respective doors of the front wall 6 also being shown. The elements that are the same or have the same effect are indicated with the same reference signs.

FIG. 5 shows, for example, another possibility for the design of the heating device 16. As shown in the drawing, this is a hot air blower, which blows the hot air through the supply pipe 20 into the lower region, i.e. the bottom region, of the drying cabinet 1. The heating may, for example, be provided by a heating sleeve 22, which is electrically powered. The bottom region may be separated by a grille 21 in this case as well.

For example, the process of drying items of clothing and equipment items may be as follows:

First, the items of clothing are suspended on the drying hangers 11 in such a way that each drying hanger 11 extends as far as possible into all parts of the items of clothing. The shoes or equipment items are suspended or placeable on the drying hangers 12 shown on the right.

The drying cabinet 1 is then closed and the heating device 16 is set to the desired temperature of about 60° C. to 80° C., preferably 65° C. to 70° C. Furthermore, the time switch 19 is operated, for example, to set the heating time until the desired drying time is reached, for example, about one hour.

At the end of the heating time, the heating device 16 switches off and the warm air blower 7 starts to blow the drying air, heated to approximately 30° C. to 60° C., preferably 40° C. to 50° C., into the air distribution ducts 13 and the drying hangers 11, 12 connected thereto. The drying time needs to be determined according to the particular conditions.

### LIST OF REFERENCE SIGNS

1 Drying cabinet
2 Bottom
3 Top
4 Left side wall
5 Right side wall
6 Front wall
7 Warm air blower
8 Air duct
9 Air inlet openings
10 Insulation
11, 12 Drying hangers
13 Air distribution duct
14 Air outlet openings
15 Coupling
16 Heating device
17 Temperature display
18 Thermostat
19 Time switch
20 Supply pipe
21 Grille
22 Heating sleeve
23 Vent opening
24 Rear wall
25 Housing

1. A drying installation, in particular for drying objects such as items of clothing and equipment items, wherein the objects are suspendable or placeable on drying hangers (11,12), wherein the drying hangers (11, 12) are arranged in a thermally insulated drying cabinet (1) which, in addition, is heatable, wherein the drying cabinet (1) comprises a bottom (2), a top (3), left and right side walls (4, 5), a rear wall (24) and an openable and closable front wall (6), wherein the drying hangers (11, 12) in the drying cabinet (1) are suppliable or are supplied with warm air by a warm air blower (7), the drying hangers (11, 12) being provided with one or more air outlet openings (14) along their extent, and wherein further a heating device (16) is arranged inside the drying cabinet (1) for heating said drying cabinet (1).

2.-3. (canceled)

4. The drying installation according to claim 1, wherein the warm air blower (7) is arranged on the outside, preferably on the top (3), and an air duct (8) is led from the warm air blower (7) to an air distribution duct (13) to which the drying hangers (11, 12) inside the drying cabinet (1) are connected or can be connected.

5. (canceled)

6. The drying installation according to claim 1, wherein the heating device (16) is provided in the bottom region of the drying cabinet (1).

7. The drying installation according to claim 1, wherein the heating device (16) is an electric heater.

8. The drying installation according to claim 1, wherein the heating device (16) is a hot air blower.

9. The drying installation according to claim 1, wherein the heating device (16) is configured for a cabinet temperature between 60° C. and 80° C., preferably 65° C. to 70° C.

10. The drying installation according to claim 1, wherein the warm air blower (7) is configured for a drying air temperature between 30° C. and 60° C., preferably 40° C. to 50° C.

11. The drying installation according to claim 1, wherein the cabinet temperature is monitored and controlled by a thermostat (18).

12. The drying installation according to claim 1, wherein a time switch (19) is provided for the switching times for the heating device (16) and optionally also for the warm air blower (7).

13. The drying installation according to claim 12, wherein the time switch is a component of an electronic controller which is set up to control the switching times for the supply of drying air to the drying hangers and for the heating device (16) and to control the drying air temperatures and cabinet temperature.

14. A method for drying and decontaminating objects such as items of clothing and equipment items, using the drying installation according to claim 1,
wherein the objects are arranged on the drying hangers (11, 12) in the cabinet and the cabinet is closed;
wherein the heating device (16) is used to bring the cabinet temperature to a temperature which is sufficient to eliminate pathogens; wherein the heating device (16) is switched off after a period of time which is sufficient to eliminate the pathogens; wherein the warm air blower (7) is switched on and the drying process is continued and completed.
